(19) United States
(12) Patent Application Publication
(10) Pub. No.: US 2018/0093036 A1
(43) Pub. Date: Apr. 5, 2018

(54) Pump Cartridge Unit Having One or More Medicament Reservoirs

(71) Applicant: Picolife Technologies, LLC, Jacksonville, FL (US)

(72) Inventor: Farid Amirouche, Highland Park, IL (US)

(21) Appl. No.: 15/832,609

(22) Filed: Dec. 5, 2017

Related U.S. Application Data
(63) Continuation of application No. 13/737,543, filed on Jan. 9, 2013.

(51) Int. Cl. A61M 5/142; A61M 5/172; A61M 5/148

(52) U.S. Cl. CPC A61M 5/142; A61M 5/172; A61M 2205/12; A61M 5/148; A61M 5/14224

(57) ABSTRACT

In one embodiment, a medicament pump cartridge may include a reservoir configured to store a medicament, wherein the reservoir may be configured to transition between an expanded configuration when filled with medicament and a collapsed configuration when empty. The medicament pump cartridge may include an electromagnetic pumping mechanism, wherein the electromagnetic pumping mechanism includes at least one magnet secured to a membrane. Further, the medicament pump cartridge may include an electromagnetic valve operably coupled to the electromagnetic pumping mechanism. The pump cartridge may be configured to allow for secured refills through compatible ports. The pump cartridge may include light indicators for ports/orifice that communicate with pump, and possess sensor volumetric feedback for continuous monitoring of the medicament volume.

### CROSS-REFERENCE TO RELATED APPLICATION

This application is a continuation of and claims priority to U.S. Nonprovisional patent application Ser. No. 13/737,543, filed Jan. 9, 2013, and titled “PUMP CARTRIDGE UNIT HAVING ONE OR MORE MEDICAMENT RESERVOIRS”, the entire contents of which are incorporated in this application by reference.

### DESCRIPTION OF THE INVENTION

### Field of the Invention

The present disclosure relates to the field of medical pumps and reservoirs. More particularly, the disclosure relates to devices and methods of storing and delivering medicament or other therapeutic agents to a patient for the management of ailments, such as, e.g., diabetes or chronic pain.

### BACKGROUND OF THE INVENTION

Conventionally, a pump and a reservoir can be two distinct portions of a medicament delivery device. The pump is responsible for causing the medicament to flow out of a reservoir into the patient, and the reservoir is configured to store the medicament. The pump may be controlled by a micro-controller. In some cases, however, the pump may be replaced with the use of a pressurized reservoir and a valve for controlling the flow of contents from the reservoir.

### SUMMARY OF THE INVENTION

Embodiments of the present disclosure relate to, among other things, a medicament pump cartridge unit having a plurality of reservoirs. Each of the embodiments disclosed herein may include one or more of the features described in connection with any of the other disclosed embodiments.

In one embodiment, a medicament pump cartridge may include a reservoir configured to store a medicament, wherein the reservoir may be configured to transition between an expanded configuration when filled with medicament and a collapsed configuration when empty. The medicament pump cartridge may include an electromagnetic pumping mechanism, wherein the electromagnetic pumping mechanism includes at least one magnet secured to a membrane. Further, the medicament pump cartridge may include an electromagnetic valve operably coupled to the electromagnetic pumping mechanism. Further, the medicament pump cartridge may include a plurality of volumetric flow rate sensor systems allowing for volumetric feedback pump control and enhanced cartridge fault detection.

In various embodiments, the medicament pump cartridge may include one or more of the following: a plurality of reservoirs; a rigid divider disposed between the plurality of reservoirs; each of the plurality of reservoirs may include a unique medicament; a port may be configured to allow refilling of the reservoir; wherein the port is configured to prevent unauthorized refilling; at least one sensor; the sensor may be configured to measure a temperature of one of the medicament disposed in the reservoir and a surrounding of the medicament pump cartridge; the sensor may be configured to measure one of a stress or strain in a wall of the reservoir; a collapse mechanism may be configured to promote collapsing of the reservoir; the collapse mechanism may include at least one resilient member; the collapse mechanism may include at least one roller configured to roll over collapsed portions of the reservoir; a case having a transparent window; a memory configured to store information; the information may include at least an age of the medicament; the reservoir may include a plurality of walls, wherein at least a portion of one of the walls is thicker relative to the remaining walls; the one of the walls includes a tapering thickness; the plurality of reservoirs may be in the form of dual shared wall collapsing reservoirs (with continuous and/or alternating drug delivery); the thickness of the shared wall between adjacent fluidic chambers may be adjusted to prevent collapsing penetration of either reservoir volume into the other; the shared wall geometry may allow for independent uniform collapsing pattern of either reservoir; the shared wall may be at an oblique angle and may allow for independent and discrete sizing of the fluidic chambers; the dual shared wall collapsing reservoir may include integrated pressure sensors built into the shared wall which allows for detection of collapsing faults and/or non-uniform collapse of either fluidic chamber; the reservoir may be formed by ultra-thin walls which aid in collapsing and minimize the required pressure differential to facilitate collapsing; a pressure differential between the inner boundary of the collapsible reservoir and the surrounding drives the collapsing feature of the reservoir and may be related to the geometry and/or wall thickness and/or material composition of the reservoir; this wall thickness may be comprised of a single material layer or may be comprised of multiple layers each offering added benefits from their material properties or characteristics, but common to all is the characteristic of being flexible; refilling of the reservoirs can be accomplished through one or more walls of the reservoir and/or fluidic chamber; the plurality of reservoirs may be connected at the shared wall level by a valve which allows for moving fluid from one to another, or for fluid mixing, for maintaining pressure across multiple cavities, for pressure relief, or may allow access from one chamber to the other chamber in the event that the pump fails; and the pump cartridge may be disposable.

In another embodiment, a medication pump cartridge may include a case defining a cavity therein, wherein a wall of the case includes a transparent window; a reservoir configured to store a medicament, wherein the reservoir is configured to transition between an expanded configuration when filled with medicament and a collapsed configuration when empty; a pump configured to facilitate delivery of medicament from within the reservoir to a patient; a memory configured to store information including an age of the medicament; a temperature sensor configured to measure a temperature of the medicament; and a collapse mechanism configured to promote collapsing of the reservoir, wherein the collapse mechanism includes at least one resilient member.

Various embodiments may include one or more of the following features: the reservoir may include a plurality of reservoirs; each of the plurality of reservoirs may include a unique medicament.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIGS. 1A-1C show perspective views and an exploded view of an exemplary pump-reservoir pump cartridge unit, in accordance with an embodiment of the present disclosure.

FIGS. 2A-2B show perspective views of an exemplary reservoir for use with a pump-reservoir pump cartridge unit of the present disclosure.

FIG. 3 shows a perspective view of an exemplary pump cartridge unit configured to allow secured refills through compatible ports and light indicators.

FIGS. 4A-4D show a perspective view, a top view, an exploded view, and a section view, respectively, of an exemplary pumping mechanism for use with a pump cartridge unit of the present disclosure.

FIGS. 5A-5C show section views of several embodiments of an exemplary flow rate sensor system for use with a pump cartridge unit of the present disclosure.

FIGS. 6A-6C show exploded perspective views of an exemplary pump cartridge unit having various sensors on a reservoir, according to further embodiments of the present disclosure.

FIGS. 7A-7D show multiple views of an exemplary reservoir, in accordance with a further embodiment of the present disclosure.

FIGS. 8A-8B show perspective views of an exemplary pump cartridge unit having a side window, in accordance with another embodiment of the present disclosure.

FIG. 9 shows an interior view of an exemplary pump cartridge unit having a temperature fuse.

FIGS. 10A-10C show perspective, section, and exploded views, respectively, of an exemplary pump cartridge unit having reservoir compression means, in accordance with a further embodiment of the disclosure.

FIGS. 11A-11D show multiple views of a pump cartridge and reservoir with resilient members and a perspective view of a reservoir with a single resilient member, in accordance with embodiments of the present disclosure.

FIGS. 12A-12D show multiple views of an exemplary pump cartridge unit, in accordance with yet another embodiment of the present disclosure.

FIG. 13A shows a view of an exemplary pump cartridge unit including a dual collapsible reservoir system, in accordance with yet another embodiment of the present disclosure.

FIGS. 13B-13C depict perspective and section views of a dual collapsing reservoir system including a shared wall defining dual fluidic chambers, in accordance with an embodiment of the present disclosure.

### DETAILED DESCRIPTION OF THE PREFERRED EMBODIMENTS

### Overview

The present disclosure relates to improving the use of medicament pumps to transport medicaments from a reservoir to a patient via, e.g., an infusion set, for delivery of insulin or other medicaments to a patient. More particularly, the disclosure relates to a pump cartridge unit of a medicament pump where the medicament reservoir and pump mechanism are combined into a single, cost-effective unit. In some embodiments, the pump cartridge unit may be a single-use disposable component configured to interact with a reusable pump or medicament distribution system. In one embodiment, the pump cartridge unit may be configured to prevent repeated uses, thereby ensuring the pump cartridge is disposable. In other embodiments, reservoirs with the pump cartridge unit may be refillable.

The present disclosure is drawn to a pump cartridge for the control and storage of medicament for a patient including, but not limited to, a first case, a second case, a pumping mechanism, a plurality of active valves, a plurality of tubing, a flow rate sensor system, an outlet, a memory module, and a plurality of reservoirs. In embodiments, the pump cartridge unit may include a single unit within which the pump and reservoirs reside. In such embodiments, the pump cartridge unit may not be connected mechanically to the control unit, but may be connected via electrical contacts for the active valves and memory modules, and a magnetic field for the main pump actuator. One or more medicaments may be stored in the reservoirs and the plurality of tubing may allow for the flow of medicaments from the reservoirs to the patient. A memory module may provide for the storage of information which includes, but is not limited to, the age of the pump cartridge, the remaining amount of medicament, the usage history of the pump cartridge, or a unique identification code. The memory module may also store, e.g., control algorithms, patient data, and/or sensor information. The reservoirs themselves may take on a number of variations such as having a thickened wall for extra support, a tapered wall, a ribbed pattern, various springs and rollers for a controlled collapse and dispensing of medicaments; a single reservoir may also be configured with a partition defining two separate medicament chambers therein, as explained below in greater detail.

In one embodiment, the disclosure includes a medicament pump and reservoir unit that improves the deficiencies in the current medicament pump designs. Embodiments of the disclosed device may include, among other things, a plurality of reservoirs, a plurality of fluid connectors, a plurality of active valves, a plurality of electrical connectors, a first pump body, a second pump body, a pump membrane, a plurality of permanent magnets, a plurality of lumens for medicament transport, a case, and a pump outlet.

The pump and reservoir may act as a single unit, which may be low-cost and disposable. The medicaments used with the disclosed devices may be entirely contained within this unit. For example, medicaments may flow from one or more reservoirs to the pump and then out of the pump cartridge. In some embodiments, the reservoirs may be disposable. In other embodiments, the reservoirs may be refillable. Still further, the reservoirs may be configured to continuously dispense medicament received from another reservoir or source (e.g., an external reservoir).

In some embodiments, one or more magnets may be utilized to actuate flow through the pump cartridge unit. A membrane may be used to displace the fluid in a chamber, and active valves controlled by electromagnetic coils may allow or prevent flow. Such configurations allow for metering of medicaments) out of the pump cartridge. The use of magnets instead of conventional geared pumps, for example, may reduce the number of moving components required to dispense medicaments to a patient.

The reservoir may be a flexible container that expands and contracts depending on the amount of fluid remaining therein. The expansion and contraction may be used to indicate to the user the remaining amount of fluids available. In addition, sensors may be operably coupled to the reservoir(s) to indicate to the pumping mechanism the amount of fluid remaining. The reservoir walls may also be configured to ensure the desired contraction shape occurs, as explained below.

Depending on patient needs, the reservoirs of the pump cartridge units disclosed herein may be pre-filled with certain medicament(s) or be unfilled. The pre-filled reservoirs may be designed in such a way so as to prevent the user from refilling in certain cases, or may be left with the option to refill medicaments using, e.g., a syringe or other suitable alternatives. For example, unfilled or empty reservoirs may be filled with medicament syringes prescribed by a physician.

The pump cartridge units described herein may be also configured to store information. For example, as alluded to above, the pump cartridge may include a memory for storing information received from, e.g., a sensor or a compatible pump interface. The information may be, but is not limited to, a unique identifying code, the remaining amount of medicaments in the reservoir of the pump cartridge, the age of the pump cartridge, the history of usage of the pump cartridge while it was connected to a pump, or any combination of those mentioned. This information may be used to inform the user when they need to replace the pump cartridge or what kinds of medicaments may be contained within the pump cartridge. In addition, the information may include, e.g., dosage information. For example, the information may include a quantity or distribution rate for a medicament. In some embodiments, the pump cartridge unit may be remotely coupled to, e.g., a physician capable of altering dosage information stored on the pump cartridge unit. For example, the pump cartridge unit may be wireless connected to the Internet for receiving such dosage instructions.

The pump cartridge unit may include one or more sensors operably coupled thereto. The sensors may be configured to monitor the conditions to which the pump cartridge may be exposed. For example, the temperature of a pump cartridge may vary depending on exposure to sunlight during, e.g., shipping. Accordingly, in some embodiments, a sensor may be incorporated into the pump cartridge unit to notify a user if the pump cartridge exceeds the acceptable temperature limits prior to or while the device is in use. Another indicator may take the form of a fuse which may become electrically separated when the temperature thresholds are exceeded. The sensor and/or fuse may be configured to communicate with the pump to prevent dispensing of the medicament stored in a pump cartridge exposed to unacceptable conditions.

### Exemplary Embodiments

A preferred embodiment of the present disclosure relates to, among other things, a pump and reservoir pump cartridge unit for the administration of therapeutic substances such as, e.g., insulin. More particularly, the present disclosure relates to devices for storing medicaments and dispensing them to a patient with, e.g., a pump driver and infusion set.

As used in connection with the following description, the term “fluid” may include, but is not limited to, a state of matter or substance (liquid or gas) whose particles can move about freely, and has neither a fixed shape nor conforms to the shape of its containers. In addition, the term “channel” may include, but is not limited to, a passage through which fluids may flow. Further, the term “medicament” may include, but is not limited to, substances used in therapy; substances used to treat, prevent, or alleviate the symptoms of disease; a medicine in a specified formulation; or an agent that promotes recovery from injury or ailment.

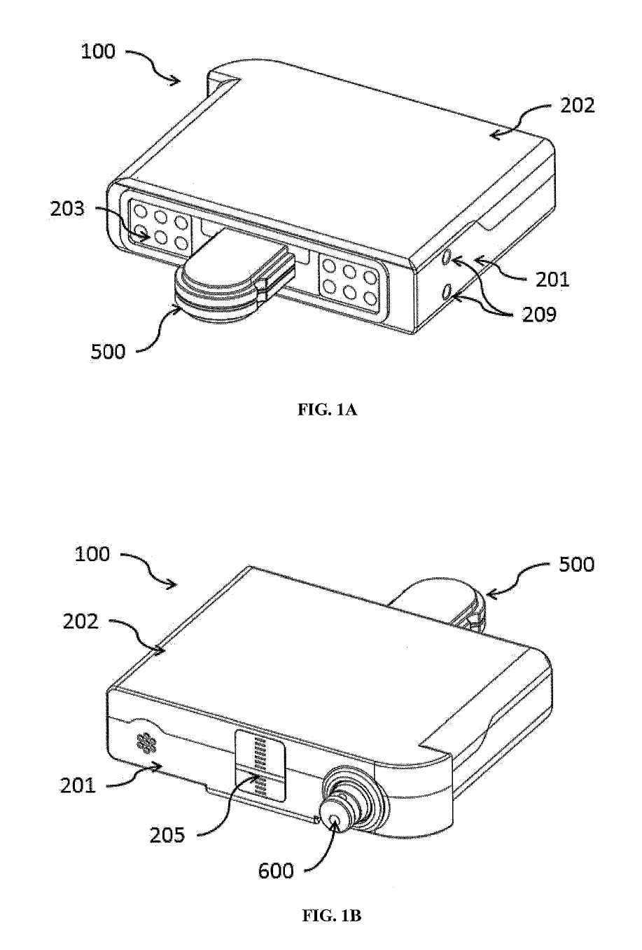

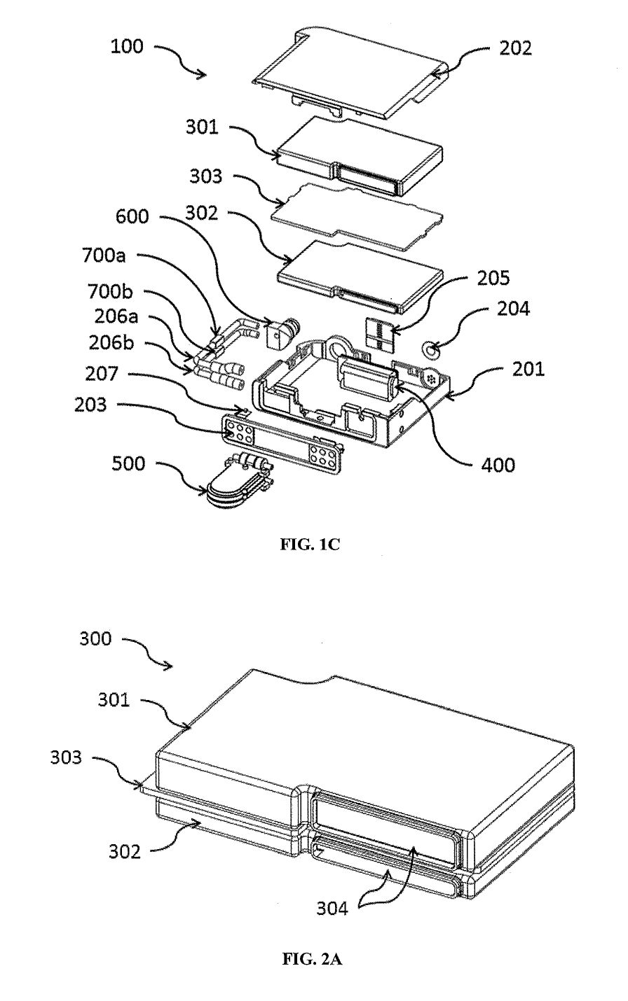

Referring now to the drawings, where like elements are designated by similar reference numerals, FIGS. 1A-1C illustrate an exemplary pump and reservoir pump cartridge unit 100, in accordance with an embodiment of the present disclosure.

In one embodiment, the pump cartridge unit 100 may include a case having a first case part 201 and a second case part 202. The first and second case parts 201, 202 may include any suitable configuration, and may be configured to cooperate to define a cavity therebetween. As shown in FIG. 1C, e.g., first and second case parts 201, 202 may include a substantially square or rectangular configuration. Case parts 201, 202 may be configured to be permanently secured to one another. Alternatively, case parts 201, 202 may be configured to be removably secured together. In embodiments where they may be secured to one another permanently, case parts 201, 202 may include tamper resistive features configured to indicate if case parts 201, 202 have been disassembled without authority. In some embodiments, a thin liner may be disposed on between the walls of a reservoir 300 (discussed below) and parts 201, 202. The liner may be configured to inhibit the transfer of heat between the exterior of the case 200 and the reservoir 300. That is, the liner may serve to thermally insulate the reservoir 300 from its surroundings.

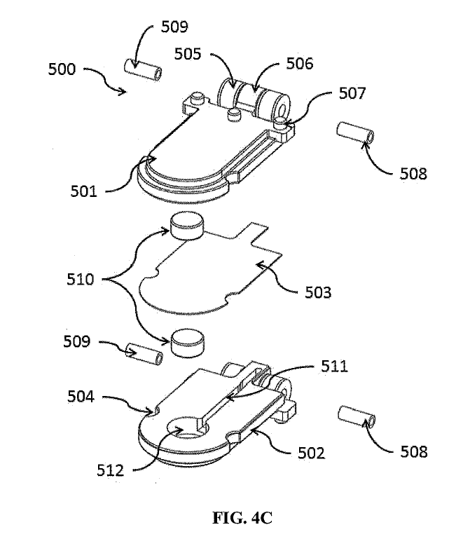

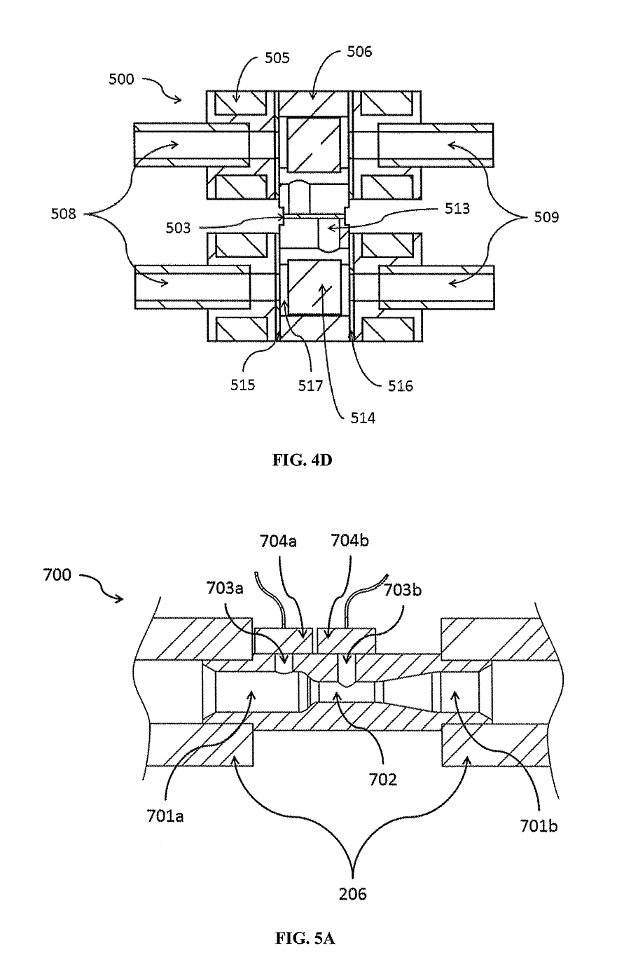

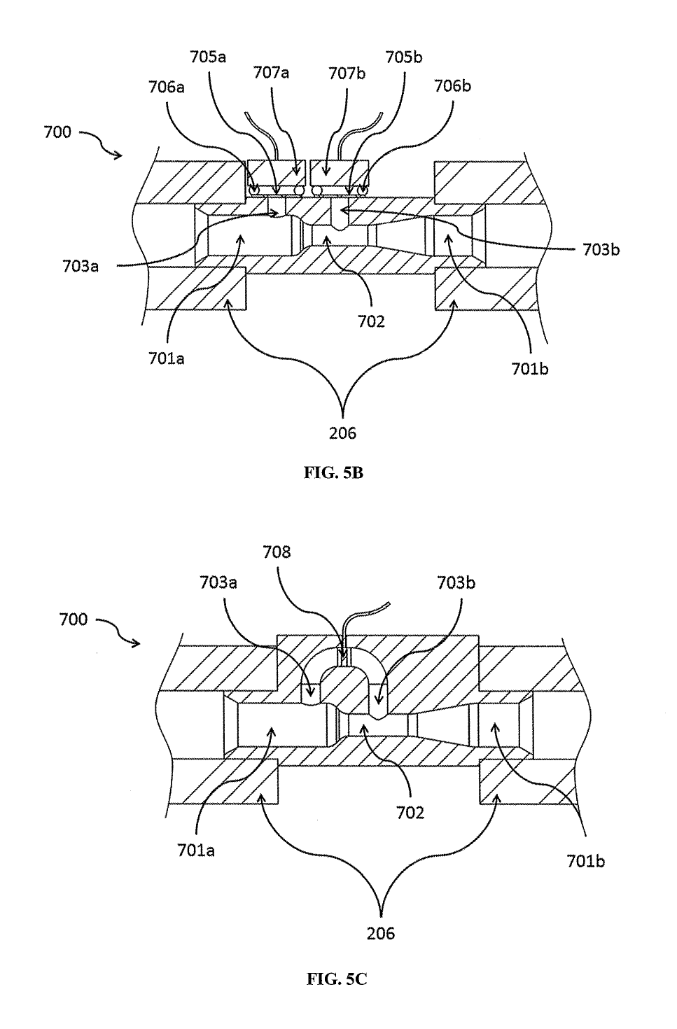

Pump cartridge unit 100 may also include one or more reservoirs 300 disposed in between case parts 201, 202. The reservoirs 300 may be configured to include one or more medicaments for delivery to a patient. The unit 100 may also include a plurality of tubes 206a, 206b (shown in, e.g., FIG. 1C) in communication with the reservoir(s) 300 and a suitable pump mechanism 500. Further, the unit 100 may include a suitable pump outlet 600 and an electronic memory/control module 203, as described below in greater detail. The outlet 600 may be connected to any suitable delivery mechanism, including, e.g., an infusion set. The pump cartridge unit 100 may also include a dual or single volumetric flow rate checking system. In some embodiments, a Hall effect sensing mechanism (not shown) may be fully integrated into the actuation mechanism. However, those of ordinary skill in the art will understand that any suitable mechanism may be used. The sensing mechanism may be configured to measure the displacement and/or travel of pump magnets 510 (shown in FIG. 4C) to calculate flow rate. For example, the displacement of pump magnets 510 during pumping may correspond to a volume of medicament pumped from a reservoir. The volume of medicament pumped from the reservoir may be then used to calculate a flow rate of medicament. In addition to this first flow rate sensing mechanism, embodiments of the present disclosure may include one or more pressure sensor mechanisms, e.g., 700a, 700b (shown in FIGS. 5A-5C), which may be used to measure the fluid flow rate near the outlet 600, as described below in greater detail. This dual volumetric flow rate sensor system may allow for a multitude of fault detections. In particular, utilizing dual flow rate measurements may make it possible to detect, e.g., air bubbles or occlusions in the medicament flow lines and/or failure of the pump mechanism. In addition, the detection of an abnormal change in pressure may indicate an issue (e.g., blockage or kink in the line) in the fluid path, which may be identified and addressed by the micro-controller. A dual volumetric flow rate sensor system may also provide a level of redundancy and error detection across the different volumetric flow rate sensing systems. In some embodiments, case parts 201, 202 may be hermetically sealed to prevent fluids from entering or exiting from pump cartridge unit 100 when assembled. In other embodiments, case parts 201, 202 may be configured to allow passage of fluids in only one direction through pump cartridge unit 100. For example, in the event of reservoir leakage, case parts 201, 202 may be coupled together in a manner that allows the leaking contents to exit the pump cartridge unit at an interface between case parts 201, 202. In another embodiment, one of case parts 201, 202 may include a membrane (e.g., a breathing membrane) configured to allow passage of only certain fluids, e.g., air or other gases, into and out of pump cartridge unit 100, so as to facilitate, e.g., deflation or collapsing of the reservoir(s) 300 therein.

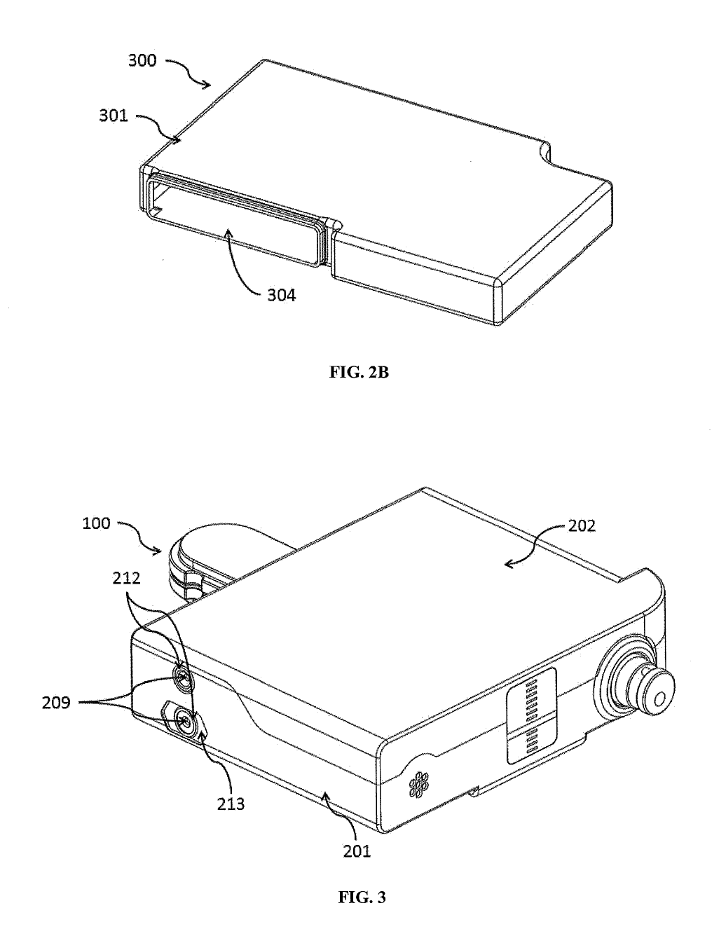

FIGS. 2A-2B show an exemplary reservoir 300 for containing a medicament for dispensing via pump mechanism 500. The reservoir 300 may include a suitable shape and configuration. In one embodiment, reservoir 300 may be an expandable pouch. In such embodiments, reservoir 300 may be made of a stretchable elastic material that will allow reservoir 300 to expand when filled with medicament to, e.g., fill the cavity formed in between case parts 201, 202. As the reservoir 300 depletes, the walls of the reservoir 300 may be configured to contract or collapse, so as ensure no air is allowed to take the place of medicament in the pouch formed by reservoir 300. For multiple fluids, two reservoirs 301, 302 may be provided. In other embodiments, however, a single reservoir 300 may be configured with a partition defining two separate medicament chambers therein. In such embodiments, the partition may be permanent, ensuring separation of the medicaments is maintained. In other embodiments, however, the partition may be selectively collapsed or destroyed to allow mixing of the medicaments just prior to use, for example. Further, although the depicted embodiments indicate that reservoirs 301, 302 may be stacked upon one another, reservoirs 301, 302 may be disposed in a side-by-side arrangement. Further, reservoirs 301, 302 may have similar or differing geometric configurations, including differing volumes.

As noted above, the reservoir 300 may be pre-filled and disposed within pump cartridge unit 100. In such embodiments, reservoir 300 may be disposable. In other embodiments, the reservoir 300 can be filled or refilled by, e.g., use of a suitable port 209 located on an outer wall of the pump cartridge unit 100 and in communication with the reservoir(s) 300. It is contemplated that reservoir 300 may be refilled with medicament that is the same as the medicament previously disposed in the reservoir 300. In addition, reservoir 300 may be refilled with a medicament that is different from the medicament previously disposed therein. For example, in a first use, the reservoir 300 may include medicament for treating diabetes. In a subsequent use, reservoir 300 may include medicament for addressing pain. As noted above, reservoir(s) 300 may be filled (or refilled) through port(s) 209 with the aid of any suitable device, such as, e.g., a syringe configured to fluidly connect to port 209.

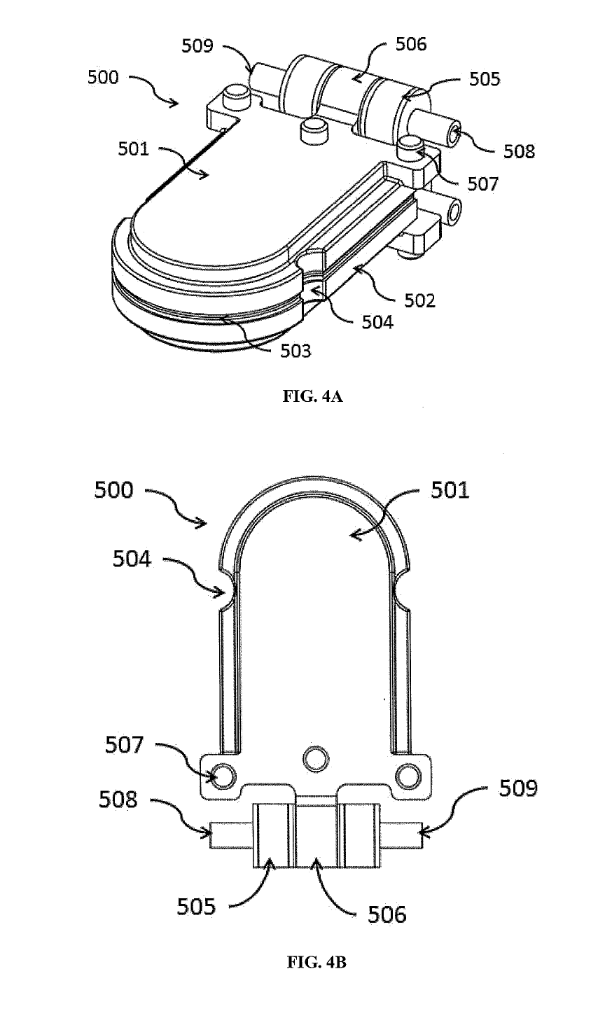

As noted above, in some embodiments, the reservoirs 301 and 302 of pump cartridge unit 100 may contain several distinct medicaments. In such embodiments, the reservoirs may be filled or refilled by a user or a healthcare provider through, e.g., ports 209. Turning now to FIG. 3, there is depicted a variety of ports 209 in first case part 201, through which reservoirs 301, 302 may be filled or refilled. Each port 209 may be associated with a suitable indicator 212 (e.g., a light emitting diode (LED)) configured to communicate with the pump and for indicating when a refill of a respective reservoir is necessary. For example, in embodiments where indicator 212 is an LED, the LED may be activated when the medicament within, e.g., reservoir is nearing depletion. In some embodiments, separate indicators 212 may be provided for indicating various levels of medicament within the reservoirs. Further, the indicator 212 is not limited to only visual indicators. In further embodiments, indicator 212 may communicate with a user via audible or tactile signals.

One or more of ports 209 may be configured to only allow refilling via authorized devices. In one embodiment, a port 209 may include a uniquely shaped fitting 213 configured to matingly receive a corresponding uniquely shaped fitting (not shown) associated with a device used to refill pump cartridge unit 100. For example, fitting 213 may include a female fitting configured to only receive a corresponding male plug of a syringe. In another embodiment, port 209 may include an electronically controlled valve (not shown) coupled to a sensor (not shown). The valve may be configured to permit refilling only if the sensor detects an authentic refilling device. Refilling devices may be provided with an electronic signal emitter, such as, e.g., an RFID emitter, which may be detected by the sensor to permit refilling. By allowing refilling via only authorized devices, it is ensured that the reservoirs of pump cartridge unit are refilled with only appropriate medicament.

As alluded to above, pump cartridge unit 100 may also include a memory/control module 203. Module 203 may include any suitable memory. In one embodiment, module 203 may include an electrically erasable programmable read-only memory. In addition to other features or capabilities, module 203 may be configured to store information relating to, among other things, the contents of reservoir 300. For example, module 203 may contain an algorithm or program configured to retrieve and store information relating to the number of times reservoir(s) 300 has been refilled and the amount of contents remaining (e.g., completely empty or partially empty) within reservoir 300 at the time of each refilling. For example, module 203 may store information indicating that a reservoir 300 was refilled 5 times, with 2 refills happening when the reservoir was only thirty percent depleted. Such information may be useful in monitoring (e.g., remotely) the use of reservoir 300 and assist in determined when a pump cartridge unit 100 may need to be replaced. In addition to being stored in module 203, such information may be stored remotely and/or on the corresponding controller.

Each pump cartridge unit 100 may also include a unique serial identifier. The identifier may be verified by a corresponding controller for identification purposes. In embodiments where pump cartridge unit 100 is intended to be single-use only (e.g., disposable), a controller may reject (e.g., inhibit medicament delivery and/or alert a user) a pump cartridge unit 100 associated with a serial identifier already used.

Further, module 203 may be configured to monitor information relating to usage and volume/units of medicament in one or more reservoir 300. This information may be used to alert a user when a refill may be needed. In addition, such information may be communicated wirelessly to a remote database via, e.g., a suitable wireless protocol enabled on pump cartridge unit 100. Such information may be used to monitor a patient's medicament usage and their consequent needs, which, in turn, may be used for marketing purposes or for automatically delivering additional medicament to users.

The port may include any suitable valve component to ensure only one-way flow of medicaments. For example, the port may include a self-sealing membrane to prevent backflow of medicament through the port. As shown in FIGS. 2A-2B, medicament stored within reservoir 300 (or reservoirs 301, 302 if multiple reservoirs are provided) may flow to the pumping mechanism 500 through an opening 304 in the reservoirs 301, 302. From there, the medicament may flow into the fluid connector 400 (shown in FIG. 1B) during the upstroke of the pump, as described in greater detail below. Fluid connector 400 may include any suitable configuration for allowing medicament within reservoir(s) 300 to flow from the reservoir(s) to pumping mechanism 500. In one embodiment, fluid connector 400 may include a channel therein for allowing the flow of medicament. In embodiments having more than one reservoir 300, however, fluid connector 400 may include any suitable number of channels corresponding to the number of reservoirs provided with pump cartridge unit 100. As shown in, e.g., FIG. 1C, fluid connector 400 may include a first opening for fluidly coupling to reservoir 300 through reservoir opening 400. In addition, fluid connector 400 may include a second opening, e.g., disposed at end of a channel therein that is opposite the first opening, configured to fluidly couple the channel to an inlet (e.g., pump inlet 508 shown in FIGS. 4A-4D) of pumping mechanism 500.

In embodiments having multiple reservoirs 301, 302, pump cartridge unit 100 may include a rigid wall 303 disposed in between reservoirs 301, 302. Wall 303 may be configured to prevent overfilling of either reservoir 301, 302 by ensuring neither reservoir expands to a size larger than a predetermined size, thereby maintaining a consistent maximum capacity for each medicament reservoir 301, 302. In other words, as explained above, reservoirs 301, 302 may be configured as expandable pouches. In embodiments having only one reservoir 300, the reservoir 300 may be filled with medicament until the reservoir 300 expands to fill a cavity defined between case parts 201, 202. That is, further expansion (and filling) of reservoir 300 may be prevented by the rigid inner walls of case parts 201, 202. In the case of embodiments having two reservoirs 301 or 302, however, if one of the reservoirs 301, 302 is inadvertently over-filled, that reservoir 301 or 302 may occupy a greater space within case parts 201, 202, thereby limiting the expansion of the other reservoir, which will in turn limit the amount of medicament that may be filled into the expansion-limited reservoir. Accordingly, the present disclosure contemplates providing a rigid wall 303 between the two reservoirs 301, 302 to appropriately limit and prevent over-expansion of either reservoir 301, 302.

Turning now to FIGS. 4A-4D, the pumping mechanism 500 may include a pair of magnetic drivers to oscillate a pair of magnets 510 connected to a membrane 503, and a plurality of pump bodies 501, 502. Pump bodies 501, 502 may include alignment pins 507 to assist with positioning pumping mechanism 500 within case parts 201, 202 of pump cartridge unit 100, as well as a locking mechanism such as, e.g., notch 504, to lock or otherwise secure the pump cartridge unit 100 inside a pump controller (not shown). More particularly, a portion of pumping mechanism 500 may include a geometric feature (e.g., notch 504) configured to interact with a corresponding geometric feature on a pump controller (not shown), such that the two geometric features may interact to ensure pump cartridge unit 100 remains operably coupled to the pump controller. An appropriate release mechanism (not shown) may be provided to facilitate decoupling of the geometric features, and, consequently, the aforementioned geometric features. The pumping mechanism 500 may include any of the exemplary pumping mechanisms disclosed in U.S. application Ser. No. 13/174,598, entitled Flow Control System for a Micropump, filed on Jun. 30, 2011, the entirety of which is incorporated herein by reference.

The pumping mechanism 500 may be operably coupled to one or more suitable valves 506. Valve 506 may include any of the exemplary valve embodiments disclosed in U.S. application Ser. No. 13/654,226, entitled Electromagnetically Actuated Non-Contact Active Microvalves and Bi-Stable Microdiverters for Fluidic Control of Micropumps and Methods Therefor, filed Oct. 17, 2012, the entirety of which is incorporated herein by reference. Valve 506 may include a bi-stable electromagnetic valve having one or more electromagnets 505 wound on geometrically custom bobbins, a plurality of silicone based seals 515, 516, a free floating permanent magnet plunger 514 therein, and high magnetic permeability stainless steel alloy pump inlet and outlet, such as, e.g., tubes 508, 509, respectively, disposed on either end of valve 506. Valve 506 may be electrically coupled to a corresponding dispensing device via electrical contacts disposed on an external surface of one of case parts 201, 202. Alternatively, pump cartridge unit 100 may include a portable power source (e.g., a rechargeable or disposable battery) for powering electromagnets 505. Once energized, the electromagnets 505 may attract permanent magnet 514 in such a manner that allows one of the seals 515, 516 of valve 506 to will restrict flow in one direction or the other depending on which electromagnet is energized, as fully explained in the aforementioned '226 application. Once the permanent magnet 514 is set to one side of the active valve or the other, the closest of the stainless steel tubes 508, 509 to the permanent magnet 514 provides the holding force required for bi-stable operation. As explained in greater detail in the '226 application, the permanent magnet 514 is flushed or pressed against an outlet seal 515 on the upstroke, which creates a temporary fluidic seal between the valve chamber 517 and the pump outlet tube 509. During this stroke, the fluid will flow from within reservoir 300 towards the pump chamber 512 through the fluid connector 400, the pump inlet tube 508 and through the valve chamber 517. The fluid then flows towards the pump chamber 512 through channels 513 and 511. On the opposite stroke, the permanent magnet 514 is flushed or pressed against the inlet seal 516, therefore creating a fluidic seal between the valve chamber 517 and the pump inlet tube 508. The fluid is pushed through channels 511 and 513 back into the valve chamber 517. The fluid then flows out of the pumping mechanism 500 through the pump outlet tube 509 and reaches the pump outlet 600 via the tubing 206.

Turning now to FIGS. 5A-5C, one of the two dual or single pressure sensor mechanisms 700a, 700b (denoted 700 here) integrated with tubing 206 near the pump outlet 600 is shown. In one embodiment, the sensor mechanisms 700 may be disposed downstream of the pump outlet 600. Medicament fluid at original pressure p1 is pushed through channel 701a of cross-sectional area A1, through channel 702 of cross-sectional area A2<A1 (i.e., small than the cross-sectional area of channel 701a), reaching pressure p2, then through channel 701b. That is, as the medicament flows through the restriction formed at the interface of channel 701b and channel 702, the fluid pressure of the medicament may experience a reduction. Once measured, the drop in pressure (p1−p2) generated by this Venturi may facilitate the calculation of the volumetric flow rate Q through tubing 206. In a first embodiment shown in FIG. 5A, pressure p1 is measured by a first pressure sensor 704a operably coupled to channel 703a, and pressure p2 may be measured by a second pressure sensor 704b operably coupled to channel 703b. Channels 703a and 703b may be filled with a biocompatible gel that insulates sensors 704a, 704b from the fluid. In addition to the biocompatible gel, any suitable material capable of performing the desired function may be employed within the principles of the present disclosure.

In another embodiment shown in FIG. 5B, pressure p1 of medicament in channel 701a may be measured by a first pressure sensor 707a and pressure p2 of medicament in channel 701b may be measured by a second pressure sensor 707b. A flexible membrane 705a may be affixed onto the opening of channel 703a (which is fluidly coupled to channel 701a) and a flexible membrane 705b may be affixed onto the opening of channel 703b, which may be fluidly coupled to channel 701b. Washers 706a, 706b and pressure sensors 707a, 707b are subsequently affixed to the top of these membranes to create small compressible air chambers [see FIG. 5B] between the membranes and the respective pressure sensors. Fluid pressure changes will cause deflections of flexible membranes 705a, 705b which will result in volumetric changes of the air chambers, which therefore will undergo changes in pressure which may be detected by the pressure sensors. The difference between the two pressure readings may directly correlate to the volumetric fluid flow of medicament through channels 701a, 701b.

In another embodiment shown in FIG. 5C, the fluid may be pushed through channels 703a, 703b and the pressure difference (p1−p2) between the medicament in channel 701a and the medicament in channel 702 may be directly measured by, e.g., differential fluid pressure sensor 708.

With renewed reference to FIG. 1C, an electronic memory module 203 may be included within pump cartridge 100. The module 203 may be configured to store information, as alluded to above. The module 203 may be placed at the interface between the pump cartridge 100 and a suitable controller (not shown), such as, e.g., the controller embodiments described in U.S. application Ser. No. 13/448,013, entitled Pump cartridge System for Delivery of Medicament, filed Dec. 1, 2011, the entirety of which is incorporated herein by reference. The interface between the controller and pump cartridge unit 100 may also provide power and data connections for actuating valve 506. In certain embodiments, pump cartridge unit 100 may include a speaker and/or other indicator (e.g., a light emitting diode or vibrator), each of which may be also powered by the power and data connections formed by interface of pump cartridge unit 100 and the controller.

Embodiments of pump cartridge unit 100 may include one or more sensors. For example, in one embodiment, a temperature sensor 207 may be operably coupled to the pump cartridge 100 for monitoring, among other things, a temperature surrounding reservoir 300 and/or a temperature of the medicament within reservoir 300. As alluded to above, if the medicament within reservoir 300 is exposed to temperatures outside a predetermined range, the medicament may be adversely affected, which may render the medicament undesired for its intended purpose. The use of a sensor 207 configured to monitor temperature of, among other things, the medicament within reservoir 300, allows a user to determine whether the medicament has been exposed to desirable or undesirable conditions, e.g., temperatures.

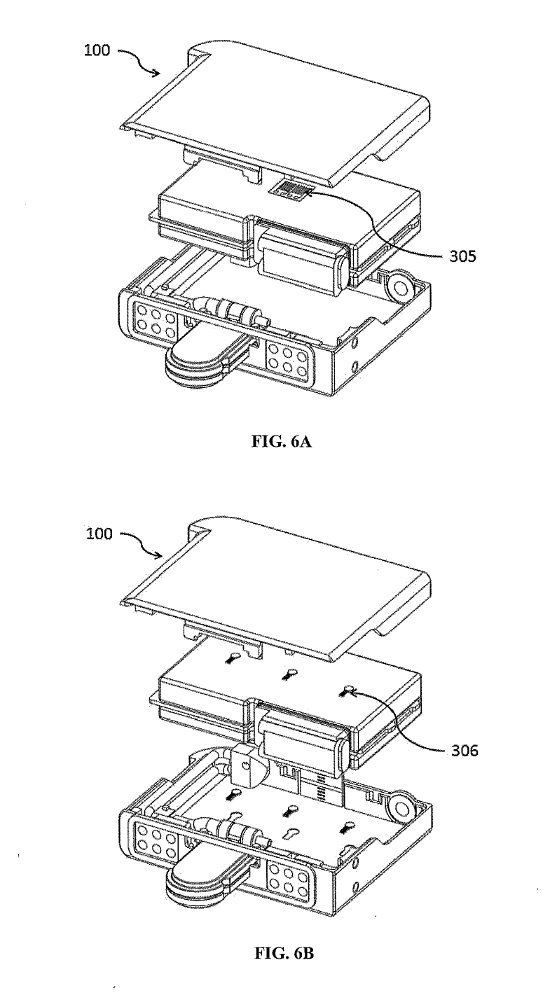

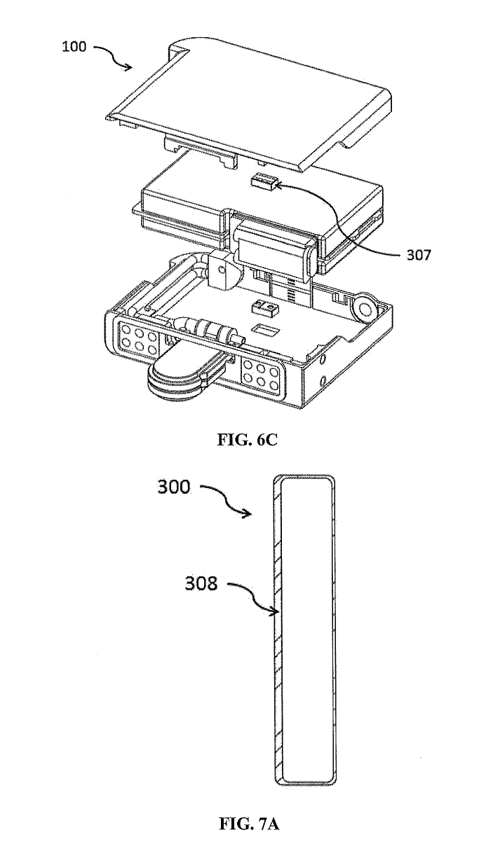

With reference to FIGS. 6A-6C, other embodiments of reservoir 300 include the use of additional or alternative sensors. For example, reservoir 300 may include a sensor 305 for continuously measuring or monitoring the amount of stress and/or strain in one or more walls of reservoir 300. For example, when reservoir 300 is completely filled with medicament, the flexible walls of reservoir 300 may be taut and therefore under a first amount of stress and/or strain. This first amount of stress and/or strain may correspond linearly to the volume of medicament in reservoir 300. As medicament is dispensed from reservoir 300, the stress/strain on the flexible walls of reservoir 300 may decrease as the volume of medicament in reservoir 300 decreases. The measurements taken from sensor 305 may be used to calculate the remaining volume of medicament in reservoir 300 during, e.g., dispensing.

In another embodiment, one or more pressure sensors 306 may be included on an external surface of reservoir 300 and/or on an internal surface of case parts 201, 202. The pressure sensors 306 may be configured to measure the forces exerted by the flexible walls of reservoir 300 on the walls of case parts 201, 202. A user filling the reservoir 300 may monitor the measurements of pressure sensors 306 to determine whether the reservoir is full. For example, as the pressure measurements from sensor 306 approach a predetermined value, the user may understand that the reservoir 300 is approaching its capacity.

In a further embodiment, pump cartridge unit 100 may include an optical sensor 307, as shown in FIG. 6C. The optical sensor 307 may be configured to measure and/or monitor a distance between one or more flexible walls of the reservoir 300 and a corresponding inner wall of parts 201, 202. As noted above, reservoir 300 may be configured to expand as it is being filled. Thus, as the reservoir 300 fills, its walls will move closer to the walls of parts 201, 202. Similarly, as the reservoir 300 empties, its walls will move away from the walls of parts 201, 202. Accordingly, by monitoring the distance between a wall of one or more parts 201, 202 and a wall of the reservoir 300, a user may be able to determine (e.g., through one or more calculations) the amount of medicament remaining in reservoir 300.

As noted above, pump cartridge unit 100 may include a control module 203. Module 203 may be configured to receive inputs from the sensors described herein and perform known calculations to determine an amount of medicament remaining in reservoir 300. Module 203 may be operably coupled to a suitable display for displaying in real-time a remaining volume of medicament within reservoir 300. The volume of medicament may be displayed in any suitable manner known in the art. For example, a gauge or numerical value of volume may be used as a display to communicate the volume of medicament remaining in reservoir 300. In addition, as the volume of medicament within reservoir 300 approaches a predetermined minimum threshold, module 203 may be configured to provide a user with a suitable indicator. For example, module 203 may cause a speaker to emit an audible alarm. In addition, or alternatively, module 203 may cause a light emitting diode to turn on, thereby providing a user with a visual indicator corresponding to a low level of medicament within reservoir 300. Further, as noted above, some embodiments of pump cartridge unit 100 may include a vibrator. In such embodiments, module 203 may cause the vibrator to create vibrations, thereby providing a tactile indicator to users suffering, e.g., visual ailments.

Other embodiments of pump cartridge unit 100 may include a shut-off fuse 208 (See FIG. 9) configured to disable an electrical connection between pump cartridge unit 100 and its corresponding controller. For example, the shut-off fuse 208 may be configured to terminate the electrical connection upon detecting temperatures exceeding a predetermined threshold. The shut-off fuse 208 may be operably coupled to the interface between unit 100 and its controller. Once the electrical connection between pump cartridge unit 100 and the controller has been disabled, the controller may be configured to prompt a user to replace pump cartridge unit 100.

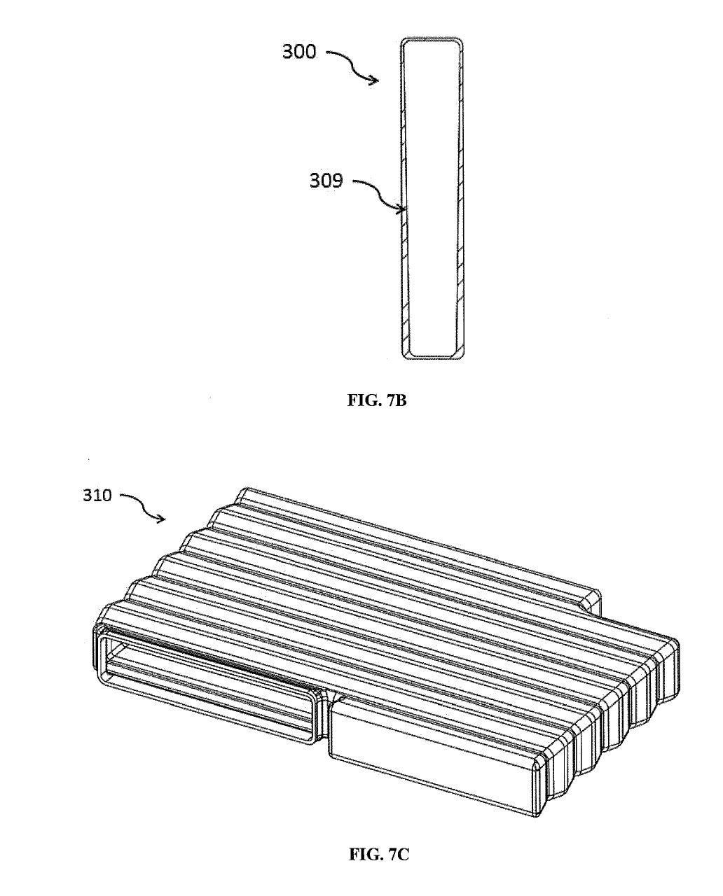

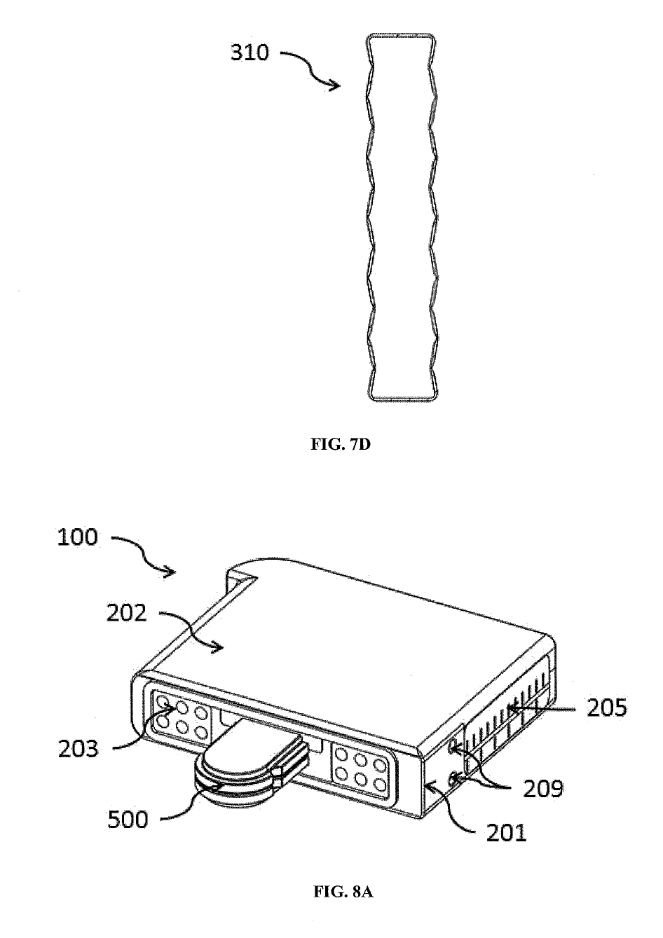

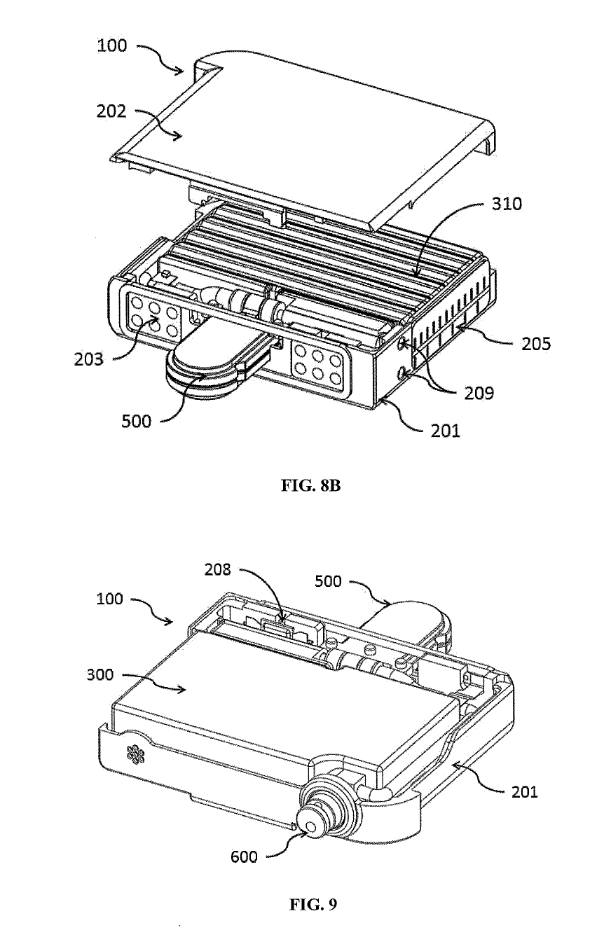

With reference now to FIGS. 7A-7B, geometrical features of the reservoir 300 can also help determine the amount of medicament remaining therein. For example, a single wall (e.g., thickened wall 308) having a thickness larger than the thickness of the remaining walls of reservoir 300 may help retain rigidity along that wall as seen in FIGS. 7A-7B. Thus, as reservoir 300 collapses, the relatively thinner remaining walls of reservoir 300 are likely to collapse before the thickened wall 308. Another embodiment of the reservoir 300 may include one or more walls 309 having a tapering thickness, which will cause the thinner sections of the wall(s) 309 to collapse before the thicker sections resulting in a controlled collapse. The controlled collapse of reservoir 300 may be readily observed through, e.g., a window 205 shown in, e.g., FIG. 1B. Window 205 may include a transparent plastic or glass disposed within an appropriately sized opening in a wall of one of case parts 201, 202. Another option for the placement of a window 205 is depicted in conjunction with the embodiments shown in FIGS. 8A-8B. In this embodiment, the transparent plastic/glass may include a plurality of graduations corresponding to volumetric measurements of medicament within reservoir 300. The reservoir 310 of FIGS. 8A-8B (also shown in FIGS. 7C-7D) may include a ribbed pattern to control the contraction of the reservoir 310. In addition, as the reservoir 310 contracts, the ribbed pattern may be visible through window 205, so that a user may be able to monitor a level of collapse of reservoir 310. In one embodiment, for example, reservoir 310 may be configured as an accordion having a plurality of expandable bellows. When in the filled configuration, each of the expandable bellows may be fully expanded. As medicament delivery begins, the bellows of reservoir 310 may begin to collapse one-by-one. The collapsing of the bellows may be visible through window 205 to assist a user in determining an approximate volume of medicament remaining within reservoir 310.

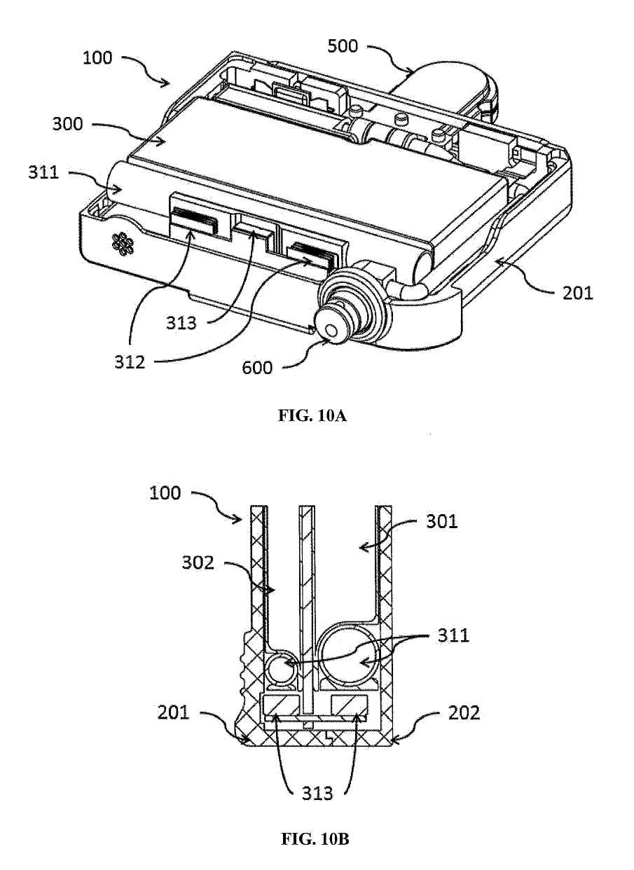

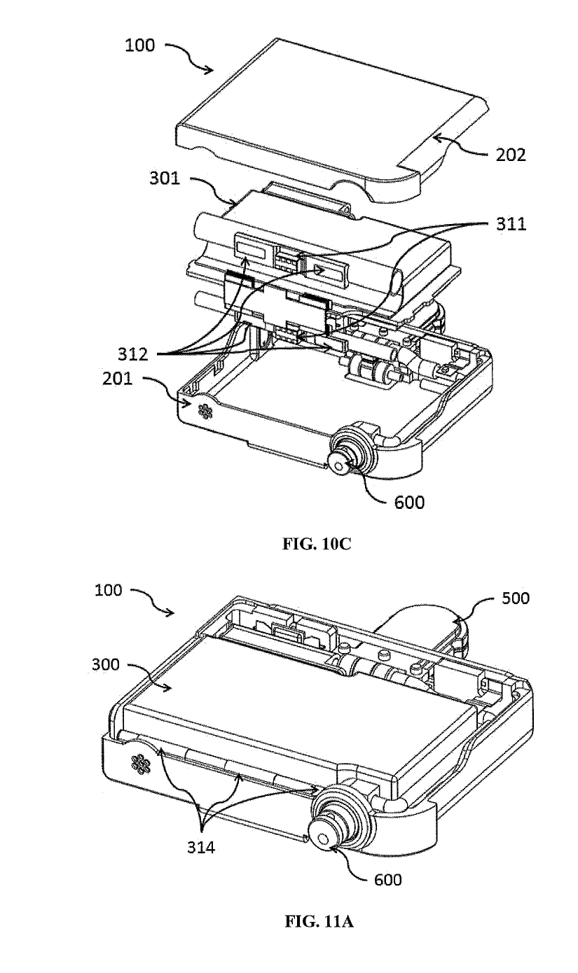

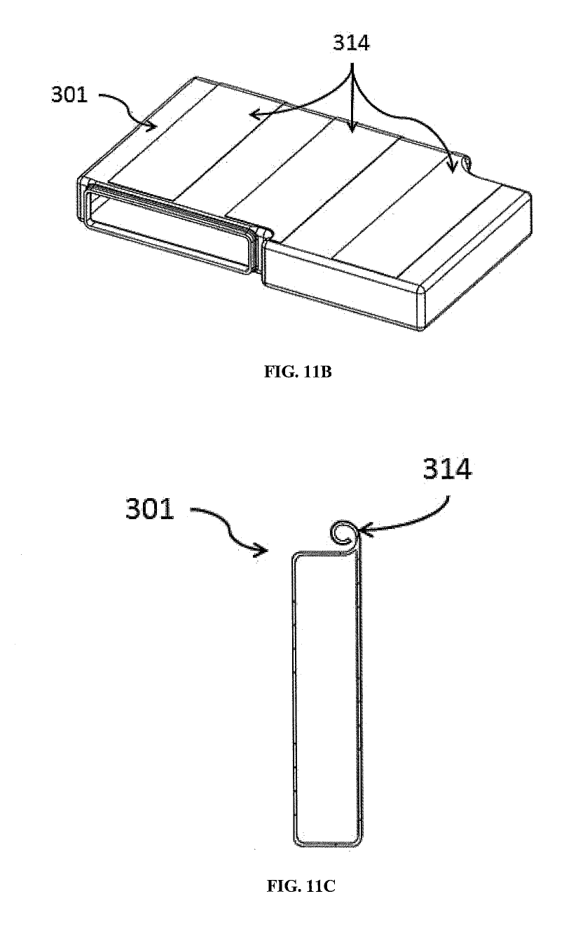

The reservoir(s) disclosed herein may be collapsed in a number of different ways. For example, the reservoirs may be configured to collapse without assistance. In some embodiments, however, controlled collapse of a reservoir 300 may be desired. In the exemplary embodiment of FIGS. 10A-10C, pump cartridge unit 100 may include a collapsing mechanism capable of controlling the collapse of a reservoir 300. The depicted embodiment includes a spring and roller system 311. The spring and roller system 311 may be configured to apply a constant or substantially constant pressure to the one or more reservoirs 300 disposed within pump cartridge 100. In one embodiment, the spring and roller system 311 may be configured to apply pressure to, e.g., a distal end of the reservoir 300, which may be affixed via adhesives to a wall of case parts 201, 202.

More particularly, as shown in FIGS. 9A-9C, pump cartridge unit 100 may include a system of resilient members, such as, e.g., springs 312 configured to provide the pressure required to collapse the reservoirs 300 as medicament is dispensed therefrom. Although the contemplated embodiment includes the use of springs 312, those of ordinary skill in the art will understand that any suitable resilient members may be used, including, but not limited to, resilient foams and the like. In addition, although the depicted embodiment only illustrates two springs 312, a greater or lesser number of springs 312 may be provided in pump cartridge unit 100. Indeed, the number of springs 312 provided in pump cartridge unit 100 may correspond to the number of reservoirs 300 in the unit 100. In addition, rather than being separate springs 312, the springs provided in pump cartridge unit 100 may include a single spring configured to apply pressure to each reservoir 300 within pump cartridge unit 100. Further, an end of each spring 312 may be secured to a wall of case part 201, 202. As shown in FIG. 9B, the springs 312 may be in a compressed configuration when reservoirs 300 are filled.

The springs 312 may be coupled to respective rollers 311. Rollers 311 may be substantially cylindrical. In addition, rollers 311 may be configured to roll within pump cartridge unit 100 via any suitable mechanism. In one embodiment, pump cartridge unit 100 may include rails upon which rollers 311 may roll. Although only two rollers 311 are depicted, the number of rollers 311 provided may correspond to the number of reservoirs 300 within pump cartridge unit 100.

In operation, the springs 312 may apply a force to rollers 311 so as to keep rollers 311 against the filled reservoirs 301, 302. As medicament is dispensed from reservoirs 301, 302, and reservoirs 301, 302 begin to collapse, the springs 312 may be configured to advance rollers 311 over the collapsed portions of reservoirs 301, 302, so as to ensure the reservoirs 301, 302 collapse uniformly across the pump cartridge unit 100. In addition, for embodiments having a window 205, as discussed above, the position of the rollers 311 may serve to indicate to a user the amount of medicament remaining in the reservoirs 301, 302. In another embodiment, pump cartridge unit 100 may include an optical sensor 313 configured to monitor a location of rollers 311. The sensor 313 may be configured to measure a distance of rollers 311 from the optical sensor, which may be used to calculate the portion of reservoir 300 already collapsed, which in turn may be used to determine the amount of medicament remaining in reservoir 300, as shown in FIG. 9A.

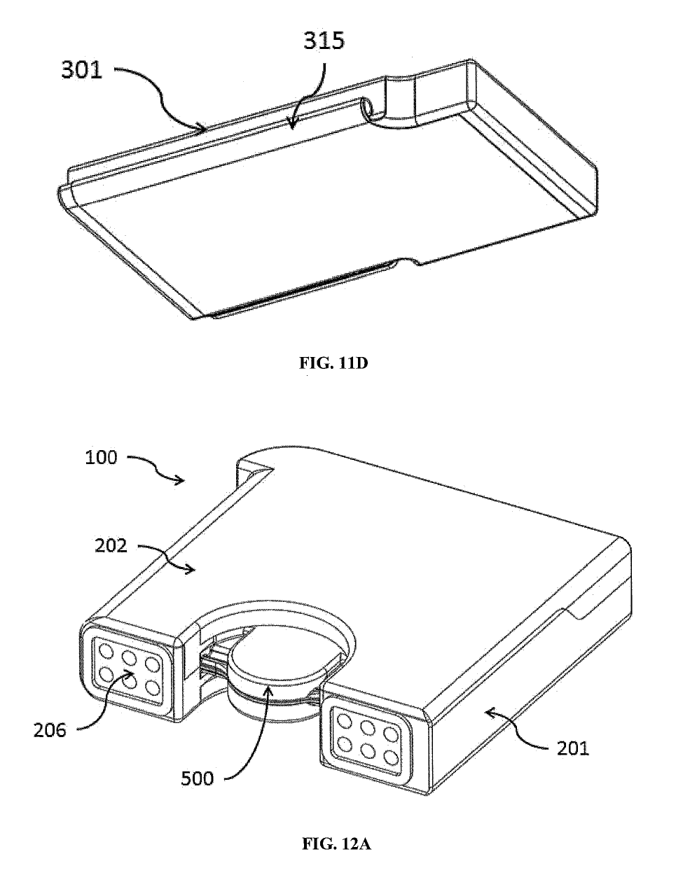

Turning now to FIGS. 11A-11D, controlled collapse of the reservoir 300 may be also accomplished by resilient members acting directly on reservoir 300. For example, as shown in FIG. 11A, reservoir 300 may be coupled to a plurality of springs 314. Each spring 314 may have a first substantially planar portion secured to a wall of reservoir 300. In addition, spring 314 may have a rolled portion extending from the planar portion. The rolled portion may be configured to be disposed adjacent the wall to which the planar spring portion is secured. Although FIG. 11B shows three springs 314, those of ordinary skill will understand that a greater or lesser number of springs 314 may be provided. Indeed, as shown in FIG. 11D, springs 314 may be replaced by a single spring 315 configured to span an entire length or width of reservoir 300. Further, although the rolled up portions of springs 314/315 are shown as disposed against a wall spanning the length of reservoir 300, the rolled up portions of springs 314/315 may be disposed against a wall spanning a width of reservoir 300. Furthermore, the rolled up portions of springs 314/315 may be disposed against any suitable wall or face of reservoir 300.

In operation, the rolled up portions of springs 314/315 may be maintained in compression against a wall of case parts 201, 202. As the reservoir empties, the springs 314/315 “unroll” to ensure the reservoir 300 collapses uniformly. In addition, the “unrolling” of springs may be viewed through a window 205 so that a user may visually observe the collapsing of reservoir 300.

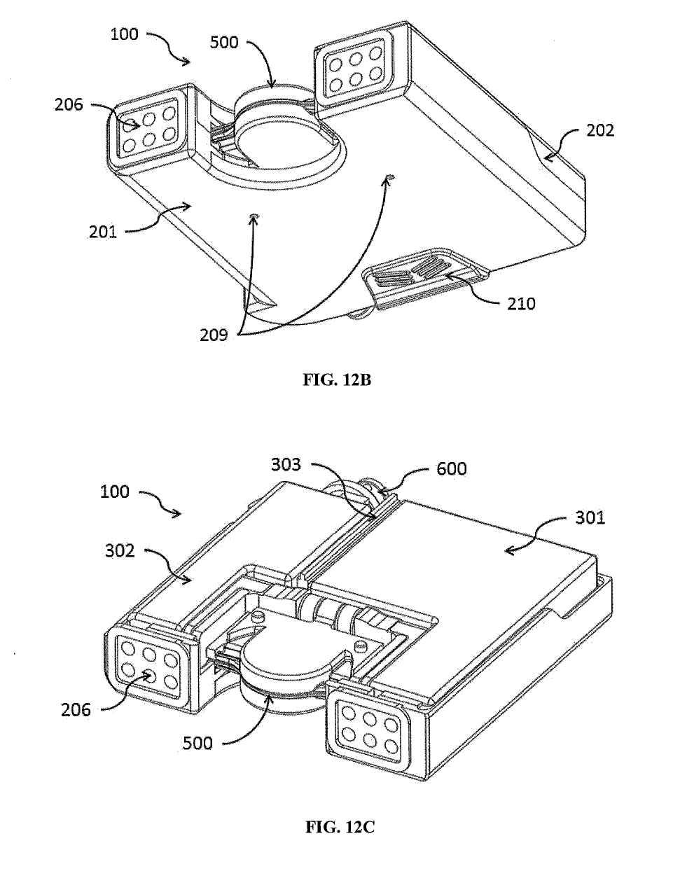

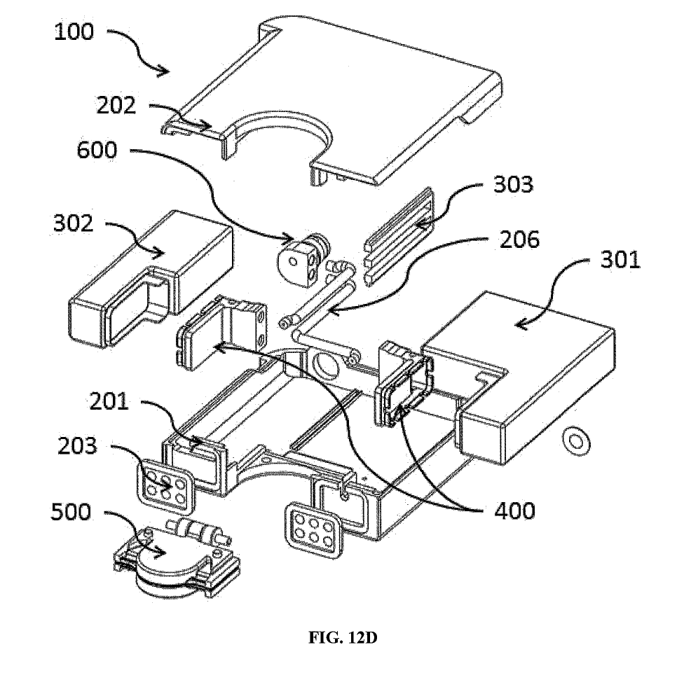

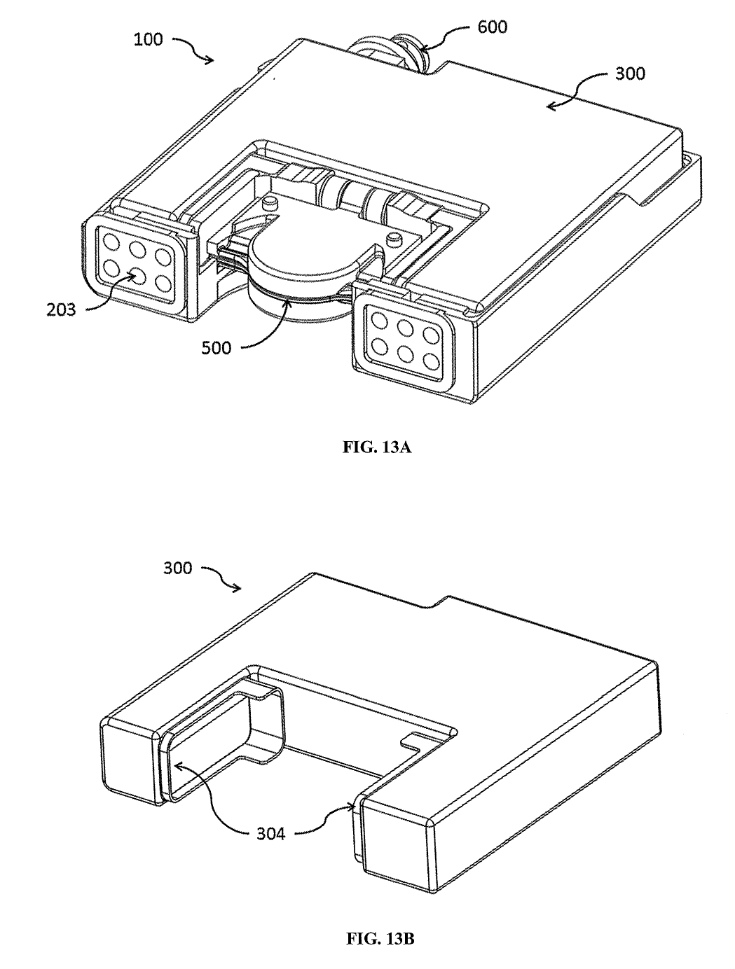

In another preferred embodiment, similar in function to the previous embodiments, the geometry of the pump cartridge unit 100 may be configured to accommodate larger reservoirs (e.g., reservoirs 301, 302) flanking the sides of the pumping mechanism 500 as seen in FIGS. 12A-12D. This embodiment provides for a more aesthetic look and a sturdier feel when inserting and removing the pump cartridge 100 from the pump controller. The reservoirs 300 provide for more capacity and are agnostic to preferences in amounts of medicaments available for the individual reservoirs 301, 302 while still utilizing a divider 303 to maintain a constant maximum level for each fluid reservoir 301, 302. In addition, as depicted in FIG. 12D, reservoirs 301, 302 may be disposed in a side-by-side arrangement. Further, the reservoirs 301, 302 may have differing configurations. For example, a volume of reservoir 301 may be different from a volume of reservoir 302. The reservoirs 301, 302 can still be refilled using ports 209 on the underside of the pump cartridge 100. A grip 210 can be seen on a first case part 201 which provides a grip for extracting the pump cartridge 100 from the pump controller. In addition, as shown in FIG. 12B, the pumping mechanism may be disposed within a cavity defined by case parts 201, 202, such that the pumping mechanism does not extend beyond an outer periphery of pump cartridge unit 100.

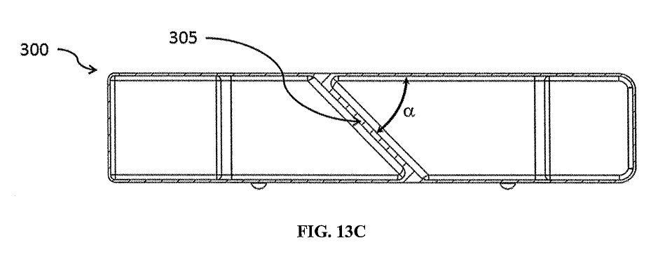

In another preferred embodiment, the geometry of the reservoir 300 may be such that it includes a dual shared wall 305 defining two separate medicament chambers and allowing for the collapse of the fluidic chambers (with continuous and alternating drug delivery) as depicted in FIGS. 13A-13C. In other words, the wall may effectively define a dual collapsible reservoir system by partitioning reservoir 300 into two fluidic chambers, both of which may be collapsible. Each of the fluidic chambers may be actuated independently and may collapse independently of one another. In addition, each fluidic chamber may be independently fluidly coupled to the medicament delivery mechanism. Also, as discussed below, both fluidic chambers may be in fluid communication.

The thickness of the shared wall 305 between the fluidic chambers may be adjusted to prevent collapsing penetration of either reservoir volume into the other. That is, the shared wall will assist in ensuring one of the fluidic chambers does not impinge on another of the fluidic chambers as it collapses during medicament delivery. It is also contemplated that geometry of the shared wall 305 may allow for independent uniform collapsing pattern of either fluidic chamber. Also, reservoir 300 may include additional walls 305 as desired. For example, in one embodiment, reservoir 300 may include two shared walls 305, which would define three collapsible fluidic chambers, thereby creating a triple collapsible reservoir system.

In one embodiment, the shared wall 305 may be at an oblique angle α, e.g. α=45°, which may allow for independent sizing of the fluidic chambers. For example, the angle of the shared wall and/or its positioning within reservoir 300 may be adjusted to affect the sizing of each fluidic chamber relative to the other. In addition, the dual shared wall collapsing reservoir 300 may include integrated sensors, e.g., pressure sensors, (not shown) integrated with the shared wall, which may facilitate detection of collapsing faults. That is, the pressure sensors may assist in detecting varying pressures within each of the fluidic chambers of reservoir 300, which may signify unequal delivery of medicament from the chambers.

The reservoir 300 may be formed by relatively ultra-thin walls which aid in collapsing and minimize the required pressure differential to facilitate collapsing of the reservoir. In addition, a pressure differential between the inner boundary of the collapsible reservoir and the surrounding environment may drive the collapsing feature of the reservoir and may be related to the geometry and/or wall thickness and/or material composition of the reservoir walls. In one embodiment, the walls of the reservoir may have a thickness less than approximately 0.25 mm, preferably less than approximately 0.13 mm, and more preferably less than approximately 0.08 mm. Walls of such thickness may be collapsed by pressure differentials lower than approximately 0.005 bar, preferably lower than approximately 0.001 bar, and more preferably lower than approximately 0.0005 bar.

The filling of the medicament chambers within reservoir 300 may be enhanced through these relatively thin walls with pressure differentials which may be lower than 0.005 bar or lower than 0.001 bar or lower than 0.0005 bar. In addition, the walls of reservoir may be made of a single material layer or may be comprised of multiple material layers, each offering added benefits from their material properties or characteristics, but common to all is the characteristic of being flexible. Furthermore, the plurality of medicament chambers may be connected through the shared wall 305 by, e.g., a valve (not shown), which may allow for moving medicament fluid from one medicament chamber to another, or for facilitate the mixing of fluids disposed in each medicament, or for maintaining pressure across multiple medicament chambers, or for pressure relief, or may allow medicament to flow from one chamber to the other chamber in the event of a pump associated with one chamber experiences failure.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

What is claimed is:

1. A medicament pump cartridge comprising:
one or more reservoirs, wherein each of the one or more reservoirs are configured to store a medicament, wherein each of the one or more reservoirs are configured to transition between an expanded configuration when filled with the medicament and a collapsed configuration when empty;
an electromagnetic pumping mechanism comprising:
a membrane,
a pair of magnets connected to the membrane,
a plurality of pump bodies,
a pair of magnetic drivers configured to oscillate the pair of magnets and the plurality of pump bodies;
a pump outlet configured to connect to a delivery mechanism, wherein the medicament passes through the pump outlet when the medicament is dispensed from the one or more reservoirs; and
a memory module configured to store information related to the medicament pump cartridge.

2. The medicament pump cartridge of claim 1, further comprising at least one sensor.

3. The medicament pump cartridge of claim 2, wherein the at least one sensor is configured to measure a temperature of one of the medicament disposed in the reservoir and a surrounding of the medicament pump cartridge.

4. The medicament pump cartridge of claim 2, wherein the at least one sensor is configured to measure one of a stress or strain in a wall of the reservoir.

5. The medicament pump cartridge of claim 4, wherein a measurement of the stress or strain in the wall of the reservoir is used to determine a level of medicament within the reservoir.

6. The medicament pump cartridge of claim 2, further comprising a first volumetric flow rate sensor system wherein the at least one sensor comprises a flow rate sensor.

7. The medicament pump cartridge of claim 1, further comprising a rigid divider disposed between the one or more reservoirs.

8. The medicament pump cartridge of claim 1, wherein each of the one or more reservoirs stores a first medicament.

9. The medicament pump cartridge of claim 1, wherein each of the one or more reservoirs stores different medicaments.

10. The medicament pump cartridge of claim 1, further comprising a port configured to allow refilling of the reservoir.

11. The medicament pump cartridge of claim 10, wherein the port is configured to prevent unauthorized refilling.

12. The medicament pump cartridge of claim 1, further comprising a collapse mechanism configured to promote collapsing of the reservoir.

13. The medicament pump cartridge of claim 12, wherein the collapse mechanism includes at least one resilient member.

14. The medicament pump cartridge of claim 12, wherein the collapse mechanism includes at least one roller configured to roll over collapsed portions of the reservoir.

15. The medicament pump cartridge of claim 1, further comprising a case having a transparent window.

16. The medicament pump cartridge of claim 1, wherein the information includes at least an age of the medicament.

17. The medicament pump cartridge of claim 1, wherein the pump cartridge is disposable.

18. The medicament pump cartridge of claim 1, wherein the pumping mechanism is configured to connect to a power source.

19. The medicament pump cartridge of claim 18, wherein the power source is located on an external device.

20. The medicament pump cartridge of claim 18, further comprising the power source.
